(19) United States
(12) Patent Application Publication
(10) Pub. No.: US 2024/0215869 A1
(43) Pub. Date: Jul. 4, 2024

(54) Sepsis Monitoring Device

(71) Applicant: Becton, Dickinson and Company, Franklin Lakes, NJ (US)

(72) Inventors: Shishir Prasad, Ramsey, NJ (US); Prasad Govindaraj, Coimbatore (IN); Sridhaar Nandakumar, Chennai (IN); Balaji Kannan, Chennai (IN); Manish Kumar, Bengaluru (IN); Ajay Suryavanshi, Pune (IN)

(21) Appl. No.: 18/093,218

(22) Filed: Jan. 4, 2023

(51) Int. Cl. A61B 5/145; A61B 5/00; A61B 5/1468

(52) U.S. Cl. CPC A61B 5/14514; A61B 5/14546; A61B 5/1468; A61B 5/6833; A61B 2010/008

(57) ABSTRACT

Provided herein is a sepsis monitoring device including: a patch having a first side and an opposing second side, where the second side is adapted to adhere to human skin; a plurality of microneedles arranged on the second side of the patch and configured to penetrate the human skin to a depth sufficient to collect interstitial fluid when the patch is adhered to the human skin, where the plurality of microneedles are configured to collect the interstitial fluid when the patch is adhered to the human skin; and a sensor in fluid communication with the plurality of microneedles and configured to detect a biomarker indicating a sepsis condition or an onset of the sepsis condition when contacted with the interstitial fluid collected by the plurality of microneedles.

### BACKGROUND OF THE INVENTION

### Field of the Invention

The present invention is directed to a device, system, and method for monitoring sepsis or the onset of sepsis.

### Description of Related Art

Sepsis is a life-threatening organ dysfunction caused by a dysregulated host response to infection. If not recognized early and managed, it can lead to septic shock, organ failure, and death. According to the World Health Organization (WHO) sepsis data, in 2017, approximately 48.9 million sepsis cases occurred, and there were approximately 11 million sepsis-related deaths worldwide. Monitoring patients and recognizing sepsis or the onset of sepsis in patients as early as possible is desired to better avoid its dangerous consequences and provide better health outcomes.

### SUMMARY OF THE INVENTION

Provided herein is a sepsis monitoring device including: a patch having a first side and an opposing second side, where the second side is adapted to adhere to human skin; a plurality of microneedles arranged on the second side of the patch and configured to penetrate the human skin to a depth sufficient to collect interstitial fluid when the patch is adhered to the human skin, where the plurality of microneedles are configured to collect the interstitial fluid when the patch is adhered to the human skin; and a sensor in fluid communication with the plurality of microneedles and configured to detect a biomarker indicating a sepsis condition or an onset of the sepsis condition when contacted with the interstitial fluid collected by the plurality of microneedles.

Also provided herein is a sepsis monitoring system including the sepsis monitoring device described herein in electrical communication with a computing device.

Also provided herein is a method for monitoring sepsis including: adhering the sepsis monitoring device described herein to skin of the patient by adhering the second side to the skin such that the plurality of microneedles penetrate the skin to a depth sufficient to collect interstitial fluid; collecting, via the plurality of microneedles, the interstitial fluid from the patient and flowing the interstitial fluid to the sensor; and detecting, by the sensor, a biomarker from the interstitial fluid, the biomarker indicating a sepsis condition or an onset of the sepsis condition.

In accordance with an embodiment of the present invention, a sepsis monitoring device includes a patch having a first side and an opposing second side, wherein the second side is adapted to adhere to human skin; a plurality of microneedles arranged on the second side of the patch and configured to penetrate the human skin to a depth sufficient to collect interstitial fluid when the patch is adhered to the human skin, wherein the plurality of microneedles are configured to collect the interstitial fluid when the patch is adhered to the human skin; and a sensor in fluid communication with the plurality of microneedles and configured to detect a biomarker indicating a sepsis condition or an onset of the sepsis condition when contacted with the interstitial fluid collected by the plurality of microneedles.

In accordance with an embodiment of the present invention, the biomarker includes at least one of a protein, a nucleic acid, and/or some combination thereof.

In accordance with an embodiment of the present invention, the nucleic acid biomarker comprises miRNA (e.g., miRNA-223).

In accordance with an embodiment of the present invention, the protein includes a C-reactive protein (CRP), Procalcitonin (PCT), Interleukin (IL)-6, and/or some combination thereof.

In accordance with an embodiment of the present invention, the biomarker includes a combination of miRNA-223 and Procalcitonin (PCT).

In accordance with an embodiment of the present invention, each of the plurality of microneedles is hollow or solid and has a length of up to 2,000 microns.

In accordance with an embodiment of the present invention, the sensor includes an electrochemical electrode comprising a conductive electrode comprising sensing materials.

In accordance with an embodiment of the present invention, the sensing materials include a hetero-bifunctional carboxyl acid- and sulfhydryl-reactive polyethylene glycol crosslinker capable of linking to the biomarker.

In accordance with an embodiment of the present invention, the sensor is configured to detect a level of the biomarker and transmit a signal to a computing device, the signal indicating the level of the biomarker.

In accordance with an embodiment of the present invention, the signal is configured to cause the computing device to display the level of the biomarker.

In accordance with an embodiment of the present invention, the sensor is configured to transmit the signal to the computing device over a short-range wireless communication connection.

In accordance with an embodiment of the present invention, the device includes a wearable electronic device.

In accordance with an embodiment of the present invention, the device includes at least one of a skin patch, a catheter care device, a catheter, and/or a tourniquet.

In accordance with an embodiment of the present invention, the plurality of microneedles are formed from a hydrogel polymer, metal, plastic, silicon, elastomer, or combination thereof.

In accordance with an embodiment of the present invention, the device is configured to monitor for the sepsis condition or the onset of the sepsis condition using the interstitial fluid from a layer of skin (e.g., the dermal layer) and without a blood sample.

In accordance with an embodiment of the present invention, a sepsis monitoring system includes a sepsis monitoring device in electrical communication with a computing device.

In accordance with an embodiment of the present invention, the computing device includes a display.

In accordance with an embodiment of the present invention, the sepsis monitoring device is adhered to skin of the patient.

In accordance with an embodiment of the present invention, the sepsis monitoring device is adhered to skin of the patient to collect interstitial fluid from the patient.

In accordance with an embodiment of the present invention, the sepsis monitoring device is adhered to skin of the patient to not collect blood from the patient.

In accordance with an embodiment of the present invention, further including a catheter inserted into the patient, the sepsis monitoring device secures the catheter to the patient. The sepsis monitoring device can be embodied onto or inside a device that secures the catheter to the patient.

In accordance with an embodiment of the present invention, wherein the system comprises a wearable electronic device.

In accordance with an embodiment of the present invention, a method for monitoring sepsis, includes adhering a sepsis monitoring device to skin of the patient by adhering the second side to the skin such that the plurality of microneedles penetrate the skin to a depth sufficient to collect interstitial fluid; collecting, via the plurality of microneedles, the interstitial fluid from the patient and flowing the interstitial fluid to the sensor; and detecting, by the sensor, a biomarker from the interstitial fluid, the biomarker indicating a sepsis condition or an onset of the sepsis condition.

In accordance with an embodiment of the present invention, further including, by the sensor, a level of the biomarker.

In accordance with an embodiment of the present invention, including transmitting, by the sensor, a signal to a computing device, the signal indicating the level of the biomarker.

In accordance with an embodiment of the present invention, further including in response to receiving the signal, displaying, by the computing device, the level of the biomarker.

In accordance with an embodiment of the present invention, further including in response to the level of the biomarker satisfying a threshold, initiating a treatment targeted at treating the patient for sepsis.

### BRIEF DESCRIPTION OF THE DRAWINGS

FIG. 1 is a schematic view of a cross-section of the layers of human skin according to non-limiting embodiments described herein;

FIG. 2 is a schematic view of a sepsis monitoring device adhered to human skin according to non-limiting embodiments described herein;

FIGS. 3A-3C are schematic views of a plurality of microneedles according to non-limiting embodiments described herein;

FIG. 4 is a schematic view of sepsis monitoring devices adhered to human skin according to non-limiting embodiments described herein;

FIGS. 5A-5B are schematic views of sensors according to non-limiting embodiments described herein;

FIGS. 6A-6C are schematic views a sepsis monitoring device and system in the form of a wearable electronic device according to non-limiting embodiments described herein;

FIGS. 7A-7D are schematic views a sepsis monitoring device and/or system in the form of a catheter care device according to non-limiting embodiments described herein;

FIG. 8 is a schematic view of a sepsis monitoring device in the form of a skin patch according to non-limiting embodiments described herein;

FIGS. 9A-9E are schematic views of a sepsis monitoring device in the form of a catheter care device and/or catheter insertion site protection device and/or a skin patch according to non-limiting embodiments described herein;

FIG. 10 is a schematic view of a catheterized patient having the catheter secured and/or catheter insertion site protected by the sepsis monitoring device from FIGS. 9A-9E according to non-limiting embodiments described herein;

FIGS. 11A-11B are schematic views of a sepsis monitoring device in the form of a catheter care device and/or catheter insertion site protection device and/or a skin patch according to non-limiting embodiments described herein;

FIG. 12 is a schematic view of a catheter having a sepsis monitoring device according to non-limiting embodiments described herein;

FIGS. 13A-13B are schematic views of a sepsis monitoring device in the form of a tourniquet according to non-limiting embodiments described herein;

FIG. 14 is a schematic view of a sepsis monitoring system according to non-limiting embodiments described herein; and

FIG. 15 is a schematic view of a sepsis treatment plan according to non-limiting embodiments described herein.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

It should be understood that any numerical range recited herein is intended to include all values and sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

The present disclosure is directed to a sepsis monitoring device, comprising: a patch having a first side and an opposing second side, wherein the second side is adapted to adhere to human skin; a plurality of microneedles arranged on the second side of the patch and configured to penetrate the human skin to a depth sufficient to collect interstitial fluid when the patch is adhered to the human skin, wherein the plurality of microneedles are configured to collect the interstitial fluid when the patch is adhered to the human skin; and a sensor in fluid communication with the plurality of microneedles and configured to detect a biomarker indicating a sepsis condition or an onset of the sepsis condition when contacted with the interstitial fluid collected by the plurality of microneedles.

The sepsis monitoring device provides numerous advantages for the diagnosis, prognosis, and treatment of sepsis. The sepsis monitoring device allows for early diagnosis of sepsis or the onset of sepsis, such that actions may be taken to improve the health outcome of the patient. The use of miRNA and/or protein biomarkers allows for diagnosis even at the systemic inflammatory response syndrome (SIRS) stage (pre-sepsis). The sepsis monitoring device may use easily accessible biomarkers in the interstitial fluid in the dermal layer of a patient to diagnose sepsis or the onset thereof. The use of these biomarkers avoids the requirement for an invasive and painful blood sample. Moreover, the sepsis monitoring device allows for quick detection of sepsis, and results can be achieved in minutes, such as less than 60 minutes, less than 30 minutes, less than 15 minutes, or less than 1 minute, such as less than 60 or 30 seconds. The sepsis monitoring device also allows for continuous monitoring of the patient such that the sepsis diagnosis can be made at the earliest possible stage in a patient being monitored. The absence of clotting factors in the interstitial fluid (as opposed to the blood) provides for easier continuous monitoring.

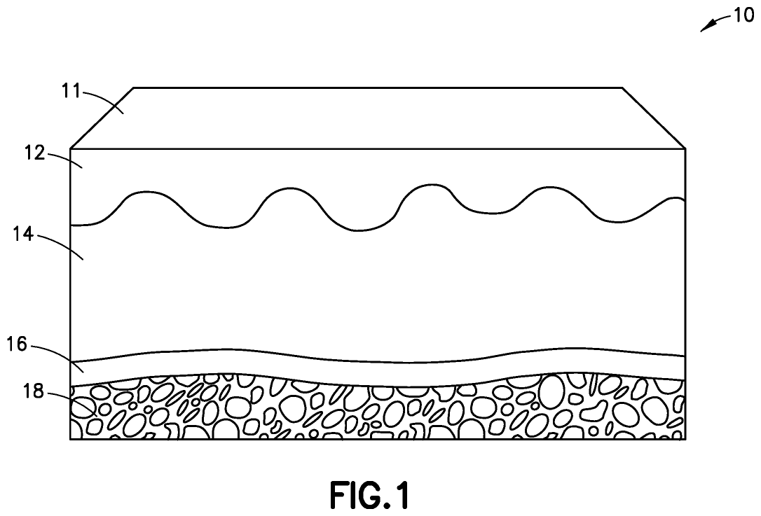

Referring to FIG. 1 a cross-section of the layers of human skin 10 is shown according to non-limiting embodiments. The human skin may have surface 11, an epidermis layer 12 beneath the surface 11, a dermal layer 14 beneath the epidermis 12, a blood circulation layer 16 beneath the dermal layer 14, and fat tissue 18 beneath the blood circulation layer 16. It will be appreciated that the sepsis monitoring device described herein may penetrate to the dermal layer 14, but not to the blood circulation layer 16. In this way, the sepsis monitoring device is configured to monitor for a sepsis condition or an onset of the sepsis condition using the interstitial fluid (from the dermal layer 14) and without a blood sample (from the blood circulation layer 16).

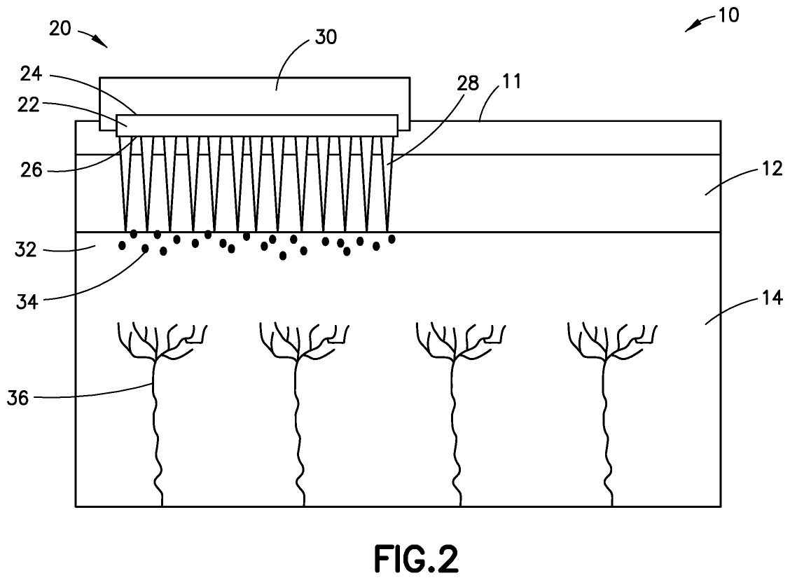

Referring to FIG. 2, a sepsis monitoring device (SMD) 20 adhered to the surface 11 of human skin is shown according to non-limiting embodiments. The SMD 20 may comprise a patch 22 having a first side 24 and an opposing second side 26. The second side 26 may be adapted to adhere to the surface 11 of human skin. For example, the second side 26 may comprise an adhesive composition suitable to securely adhere the second side 26 to the surface 11 of human skin. Additionally, or alternatively, the patch 22 may be adhered to the surface 11 of human skin by any other mechanism, such as by being taped to the surface 11, or by the microneedles 28 described hereinafter functioning to secure the patch 22 to the surface 11 of human skin, or by using a band or belt.

The SMD 20 may comprise a plurality of microneedles 28 arranged on the second side 26 of the patch 22. The microneedles 28 may be configured to penetrate the human skin to a depth configured to collect interstitial fluid (ISF) 32 when the patch 22 is adhered to the surface 11 of human skin. The microneedles 28 may penetrate to the dermal layer 14 to collect ISF 32 surrounding cells in the dermal layer 14. The microneedles 28 may continuously extract ISF 32 to enable continuous monitoring of the patient. The microneedles may extract from 0.5-10 μL of ISF 32 per minute, such as 1-5 μL per minute. The ISF 32 may comprise biomarkers 34 capable of indicating a sepsis condition or an onset of the sepsis condition. ISF 32 may be a suitable substitute for a blood sample due to its rich source of biomarkers which strongly correlate with blood dynamics. For example, the ISF 32 may have a >98% protein biomarker overlap and a >95% nucleic acid overlap with that of blood biomarkers. The microneedles 28 may not penetrate to the depth of the blood circulation layer 16 (from FIG. 1) and may not penetrate to the depth of pain receptors 36, such that use of the SMD 20 comprising the microneedles 28 may be painless to a patient and may not rupture blood vessels.

With continued reference to FIG. 2, the SMD 20 may comprise a sensor 30 in fluid communication with the microneedles 28. The sensor 30 may be configured to detect the biomarkers 34 indicating the sepsis condition or the onset of the sepsis condition when the sensor 30 is contacted with ISF 32 collected by the microneedles 28.

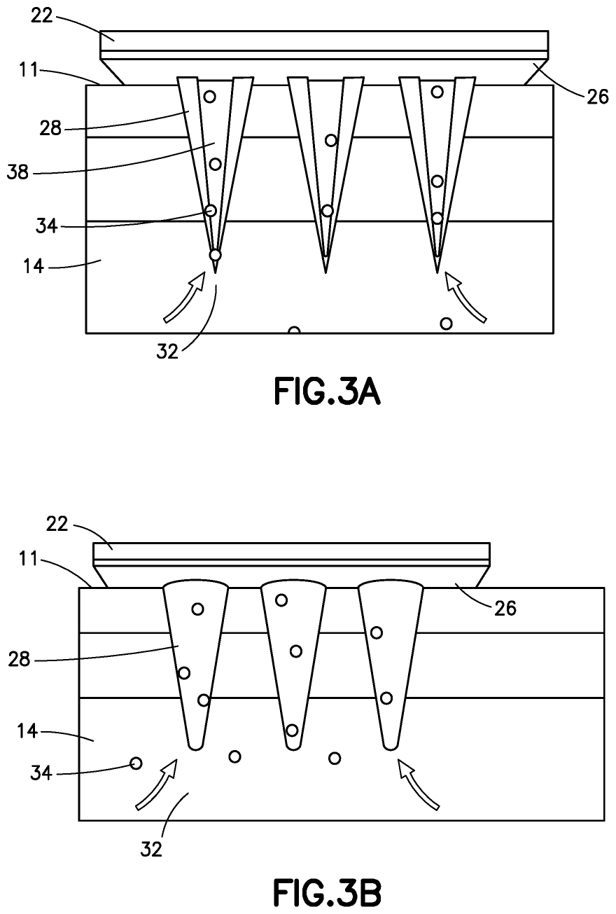

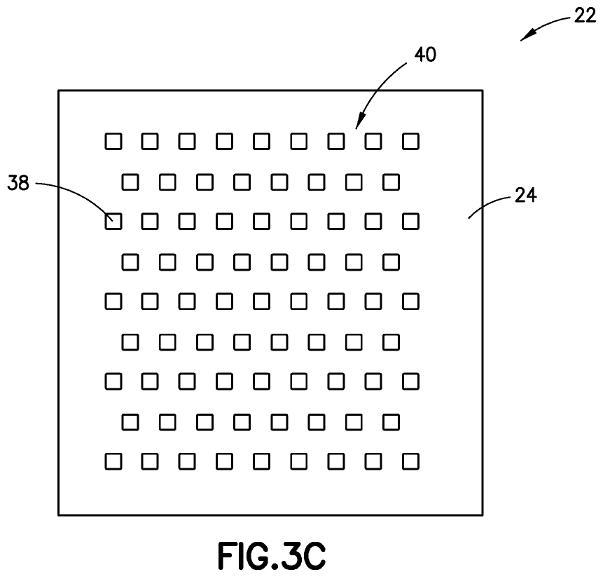

Referring to FIGS. 3A-3C, non-limiting examples of the patch 22 comprising microneedles 28 are shown. As shown in FIGS. 3A-3B, the microneedles 28 may be arranged on the second side 26 of the patch 22 and may penetrate the surface 11 of the human skin when the patch 22 is adhered thereto. The microneedles 28 may penetrate to the depth of the dermal layer 14. The microneedles 28 may have a length of up to 2,000 microns, such as up to 1,000 microns, so as to penetrate to the desired depth and to avoid causing pain to the patient. For example, the microneedles 28 may have a length of from 25-2,000 microns or from 300-1,000 microns.

With continued reference to FIG. 3A-3B, the microneedles 28 may collect ISF 32 from the dermal layer 14, and the collected ISF 32 may comprise the biomarkers 34. The microneedles 28 may comprise a hollow tube 38 to collect the ISF 32 and biomarkers 34 and transport the same from the dermal layer 14, through the microneedles 28, to the sensor 30. In some non-limiting embodiments, the microneedles 28 may be porous defining hollow tubes 38. The microneedles 28 may comprise vacuum-assisted microneedles, paper-based sampler microneedles, and/or swollen hydrogel microneedles. The ISF 32 and biomarkers 34 may be collected by the microneedles 28 based on a negative pressure and/or a capillary force. The ISF 32 and biomarkers 34 may be collected by the microneedles 28 by fluid diffusion and/or material absorption. The microneedles 28 may be formed from a hydrogel and/or coated with a hydrogel, such as a polymeric crosslinking network containing hydrophilic groups, configured to aid in collection of the ISF 32 and biomarkers 34. The microneedles may be formed from a non-dissolving rigid or solid material. The microneedles 28 may be formed from metal, plastic, or silicone.

Referring to FIG. 3C, the first side 24 of the patch 22 is shown. The first side 24 may define an array 40 of microneedles 28 (needles themselves not shown), and the first side 24 may define a plurality of hollow tubes 38 defining the microneedles 28 and placing the microneedles 28 in fluid communication with the sensor 30 (not shown).

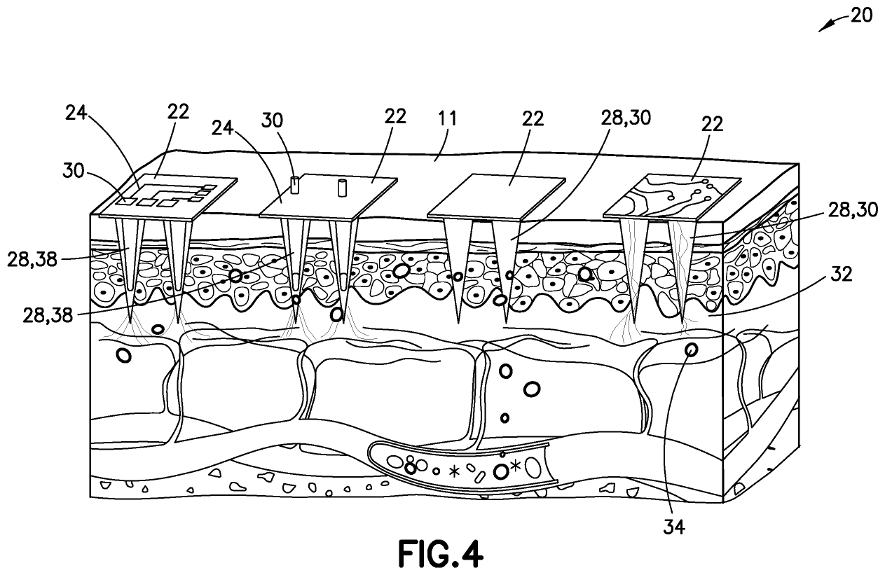

Referring to FIG. 4, several non-limiting examples of SMD 20 are shown adhered to the surface 11 human skin. In particular, four (a-d) SMDs 20 are shown, each configured to collect ISF 32 and biomarkers 34. In all four (a-d) SMDs 20, microneedles 28 penetrate the surface 11 of human skin to collect ISF 32 and biomarkers 34, but each embodiment may have a different arrangement of the microneedles 28 and sensor 30. In embodiment (a) of the SMD 20, the microneedles 28 comprise a hollow tube 38 for flowing ISF 32 and biomarkers 34 to the sensor 30. The sensor 30 in this example is arranged on the first side 24 of the patch 22. In embodiment (b) of the SMD 20, the microneedles 28 comprise a hollow tube 38 for flowing ISF 32 and biomarkers 34 to the sensor 30. The sensor 30 in this example is at least partially arranged in the hollow tube 38 of the microneedle 28 and protrudes from the first side 24 of the patch 22. In embodiment (c) of the SMD 20, the microneedles 28 and the sensors 30 are the same component. In this way, the microneedle sensors 28, 30 penetrate the surface 11 of the skin, and the ISF 32 and biomarkers 34 directly contact the microneedle sensors 28, 30 beneath the surface 11 of the skin. In embodiment (d) of the SMD 20, the microneedles 28 and the sensors 30 are the same component by metallization of the microneedles 28 as dry electrodes. In this way, the microneedle sensors 28, 30 penetrate the surface 11 of the skin, and the ISF 32 and biomarkers 34 directly contact the microneedle sensors 28, 30 beneath the surface 11 of the skin.

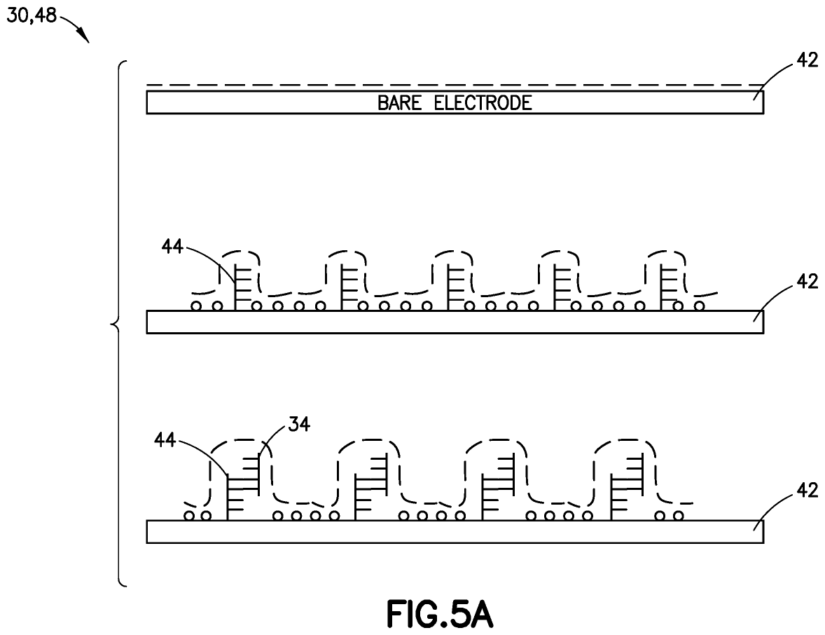

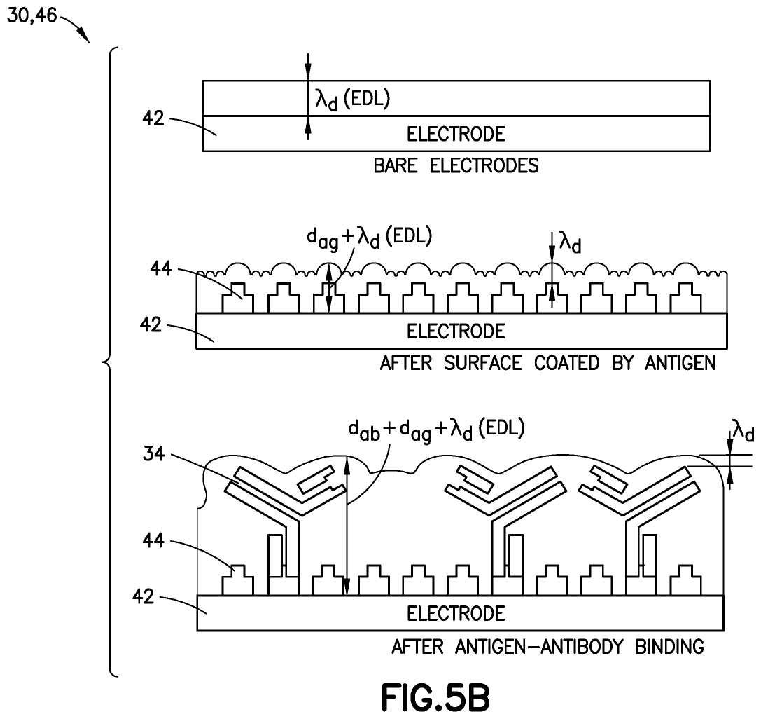

Referring to FIGS. 5A-5B sensors 30 are shown according to non-limiting embodiments. The sensor 30 of FIG. 5A shows a micro RNA (miRNA) biomarker detector 48, while the sensor 30 of FIG. 5B shows a protein biomarker detector 46. It will be appreciated that a sensor 30 that is both a miRNA biomarker detector 48 and a protein biomarker detector 46 may be used in some non-limiting embodiments.

The sensor 30 may be in fluid communication with the microneedles 28 (not shown) and be configured to detect a biomarker 34 indicating a sepsis condition or an onset of a sepsis condition when contacted with ISF 32 (not shown) containing the biomarker 34. As used herein, a sepsis condition or an onset of a sepsis condition refers to a sepsis diagnosis or a precursor to sepsis. These concepts include sepsis and septic shock, as well as their precursor: systemic inflammatory response syndrome (SIRS). The sensor 30 may be capable of detecting the sepsis condition or onset thereof by analyzing relevant biomarkers 34. The relevant biomarkers 34 for diagnosing sepsis or its onset may comprise any suitable protein, nucleic acid (including miRNA), and/or combination thereof. The biomarker 34 may comprise lactate. Suitable miRNA may include miRNA-223, miRNA-126, miRNA-146, and/or some combination thereof, particularly miRNA-223. The miRNA-223 biomarker is capable of distinguishing between infection induced-SIRS and non-infection induced-SIRS. Suitable proteins may include a C-reactive protein (CRP), Procalcitonin (PCT), Interleukin (IL)-6, and/or some combination thereof. In some non-limiting embodiments, a combination of miRNA and protein biomarkers 34 may be used. For example, a miRNA-223 miRNA biomarker 34 may be used in combination with a PCT protein biomarker 34.

FIG. 5A shows the formation and use of the sensor 30 as a miRNA biomarker detector 48. The sensor 30 may comprise an electrochemical electrode comprising a conductive electrode 42 comprising sensing materials 44. In step (a), a bare conductive electrode 42 is shown. In step (b), the conductive electrode 42 is coated in the sensing material 44. The sensing material 44 may be configured to interact with relevant biomarkers 34, such as relevant miRNA biomarkers 34. The sensing material 44 may comprise a DNA probe adapted to link to a specific miRNA biomarker 34 in the ISF 32. The sensing material 44 may comprise a spacer molecule. The spacer molecule may comprise, for example, 6-mercaptohexanol (MCH). The spacer molecule may suppress adsorption of DNA on a bare surface (e.g., a gold surface) of the conductive electrode 42 and encourage the dsDNA to adopt a vertical orientation on the surface, thus preventing or minimizing non-specific binding on DNA/RNA sensors. The DNA probe-modified conductive electrode 42 may be treated with a solution containing the spacer molecule to minimize non-specific binding and control the density of the DNA probe on the surface of the conductive electrode 42 by providing a spacer therebetween to increase hybridization efficiency. In step (c) the sensing material 44 linked to the specific miRNA biomarker 34 is shown which enables the sensor 30 to monitor and determine the sepsis condition or onset of the sepsis condition.

FIG. 5B shows the formation and use of the sensor 30 as a protein biomarker detector 46. The sensor 30 may comprise an electrochemical electrode comprising a conductive electrode 42 comprising sensing materials 44. In step (a), a bare conductive electrode 42 is shown. In step (b), the conductive electrode 42 is coated in the sensing material 44. The sensing material 44 may be configured to interact with relevant biomarkers 34, such as relevant protein biomarkers 34. The sensing material 44 may comprise an antigen probe adapted to link to a specific protein (e.g., antibody) biomarker 34 in the ISF 32. In some non-limiting examples, the sensing material 44 may comprise a hetero-bifunctional carboxyl acid- and sulfhydryl-reactive polyethylene glycol (PEG) crosslinker capable of linking to the protein biomarker 34 (e.g., IL-6) (an antibody/antigen link). In step (c) the sensing material 44 linked to the relevant protein biomarker 34 is shown which enables the sensor 30 to monitor and determine the sepsis condition or onset of the sepsis condition.

With continued reference to FIGS. 5A-5B, the sensor 30 may comprise an alternating current (AC) electrokinetic capacitive sensor. The sensor 30 may be biocompatible so as to be suitable for use in contact with human patients. The sensor 30 may analyze the ISF 32 in contact therewith to determine a level of relevant biomarkers 34 in the ISF 32. The presence of the relevant biomarkers 34 linked to the sensing material 44 may be detected by the sensor 30 based on an impedance analyzer detecting a change in capacitance. The change in capacitance may correlate to a level of the biomarker 34, such that the sensor 30 is configured to detect a level of the biomarker 34. The sensor 30 may detect the biomarker 34 within 30-60 seconds. It will be appreciated that the sensor 30 may use any other mechanism for determining a level of a biomarker 34 relevant for indicating the sepsis condition or the onset thereof. The sensor 30 may also sense antibiotic levels in the ISF 32 to provide actionable treatment data based on the sensed antibiotic levels. An antibiotic dosage may be adjusted based on the sensed antibiotic levels as per treatment requirements.

The present disclosure is also directed to a sepsis monitoring system comprising the sepsis monitoring device as described herein in electrical communication with a computing device.

As used herein, the term “computing device” or “computer device” may refer to one or more electronic devices that are configured to directly or indirectly communicate with or over one or more networks. The computing device may be a mobile device, a desktop computer, or the like. The computing device may be a wearable device, as a smartwatch. Furthermore, the term “computer” may refer to any computing device that includes the necessary components to receive, process, and output data, and normally includes a display, a processor, a memory, an input device, and a network interface.

As used herein, the terms “electrical communication” and “communicate” refer to the receipt or transfer of one or more signals, messages, commands, or other type of data. For one unit (e.g., any device, system, or component thereof) to be in communication with another unit means that the one unit is able to directly or indirectly receive data from and/or transmit data to the other unit. This may refer to a direct or indirect connection that is wired and/or wireless in nature. Additionally, two units may be in communication with each other even though the data transmitted may be modified, processed, relayed, and/or routed between the first and second unit. For example, a first unit may be in communication with a second unit even though the first unit passively receives data and does not actively transmit data to the second unit. As another example, a first unit may be in communication with a second unit if an intermediary unit processes data from one unit and transmits processed data to the second unit. It will be appreciated that numerous other arrangements are possible.

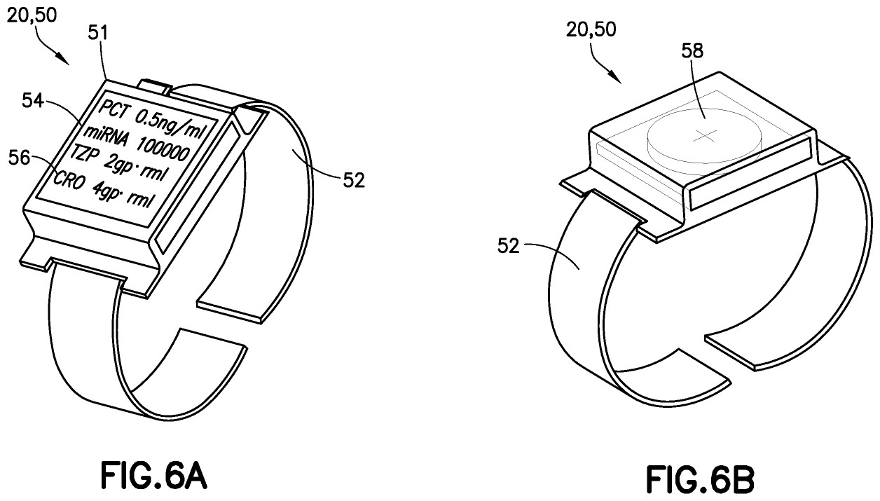

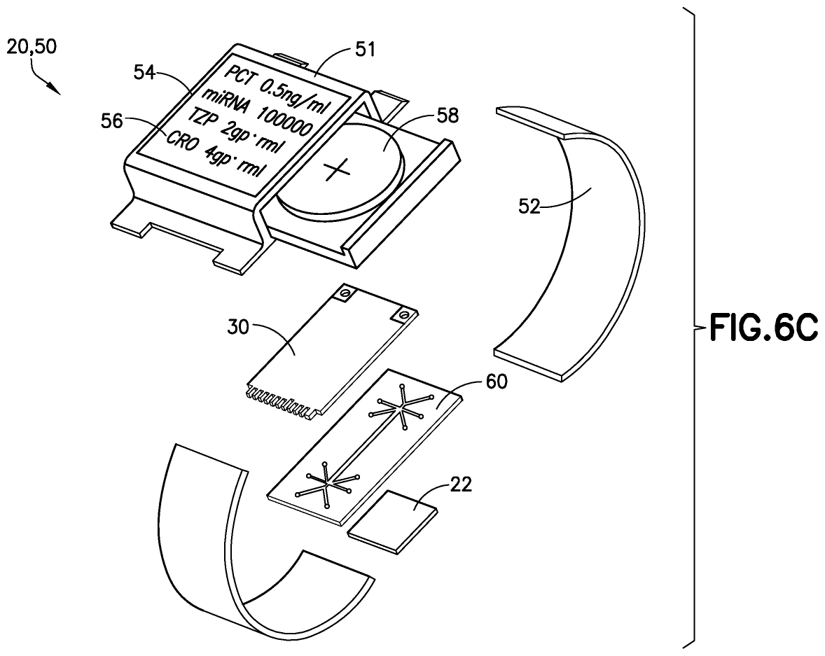

Referring to FIGS. 6A-6C a sepsis monitoring device (SMD) and a sepsis monitoring system (SMS) 20, 50 are shown in the form of a wearable electronic device. The computing device 51 in this non-limiting example is a smartwatch, although it will be appreciated that the SMD 20 may be used in conjunction with any suitable computing device 51 to form the SMS 50. The computing device 51 may be a patient's smartphone. For example, the computing device 51 may comprise a nursing station or any other computing device 51 used by a healthcare professional. The computing device 51 in the form of the smartwatch may comprise a wristband 52 enabling the SMS 50 to be wearable. The computing device 51 may also comprise a display 54 comprising a graphical user interface on which data may be displayed in a format readable by a user. The display 54 may display biomarker data 56 comprising the biomarker sensed by the sensor 30 and a level of the sensed biomarker and/or medications (antibiotics). For example, the display in FIG. 6A shows a level of PCT, miRNA, piperacillin/tazobactam (TZP), and ceftriaxone (CRO). The computing device 51 may comprise a battery 58 to power the computing device 51 and/or the SMD 20 (e.g., the sensor 30 thereof), although the computing device 51 may use any suitable power source.

Referring to FIG. 6C, the SMD 20 may be integrated with the computing device 51 to form the SMS 50. In this non-limiting example, the SMD 20 may be arranged on a portion of the smartwatch adapted to be in contact with the skin of the patient (wearer). The SMD 20 may comprise the patch 22 as previously described having microneedles 28 (not shown) that penetrate the wearer's skin when the computing device 51 (watch) is worn. The SMD 20 may comprise the sensor 30 as previously described. The SMD 20 may comprise a microfluidic device 60 configured to flow the ISF 32 collected by the microneedles 28 to the portion of the sensor 30 configured to analyze the ISF 32 for the sepsis condition or onset thereof. The microfluidic device 60 may comprise channels to direct the ISF 32 to the relevant portion of the sensor 30.

The SMD 20 may be configured to detect the level of the biomarker(s) 34 indicating a sepsis condition or an onset of the sepsis condition and transmit a signal to the computing device 51, which signal indicates the level of the biomarker 34. The SMD 20 may transmit the signal to the computing device 51 by generating a message containing the level of the biomarker 34 and communicate the message to the computing device 51. The signal transmitted to the computing device 51 may cause the computing device 51 to display the biomarker data 56 on the display 54. The SMD 20 may be configured to communicate the signal to the computing device 51 over a short-range wireless communication connection (e.g., an NFC communication connection, an RFID communication connection, a Bluetooth® communication connection, etc.), although any other form of wired or wireless communication connection may be used.

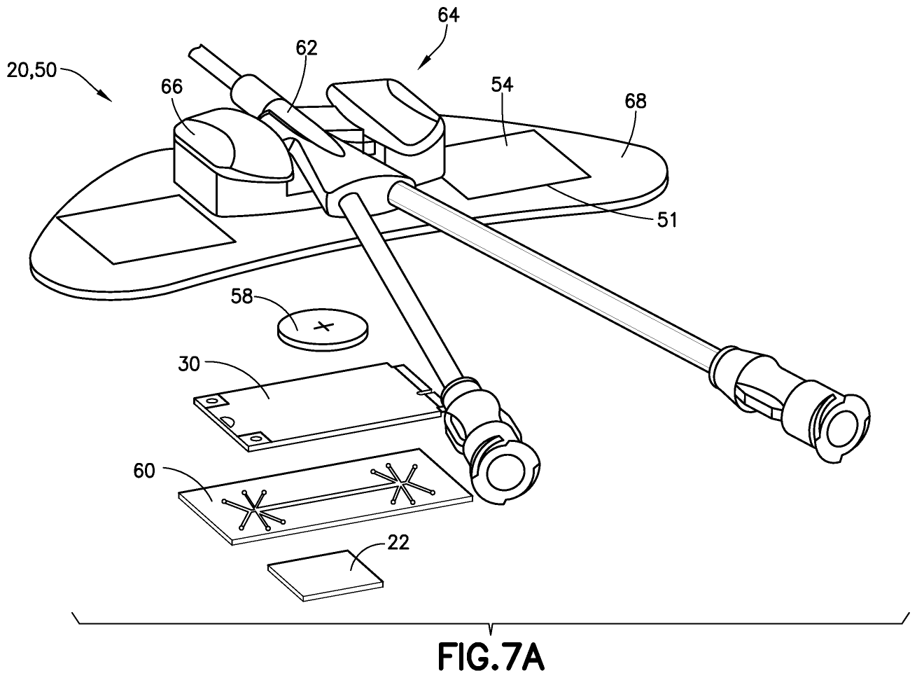

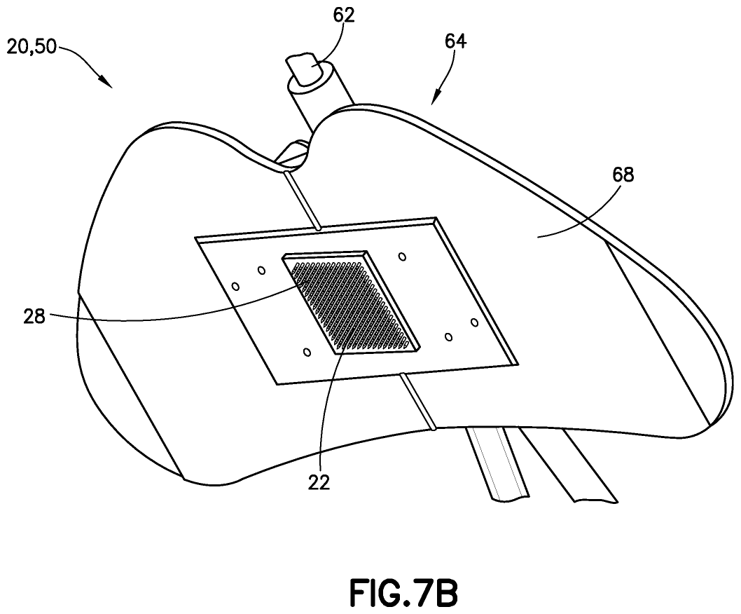

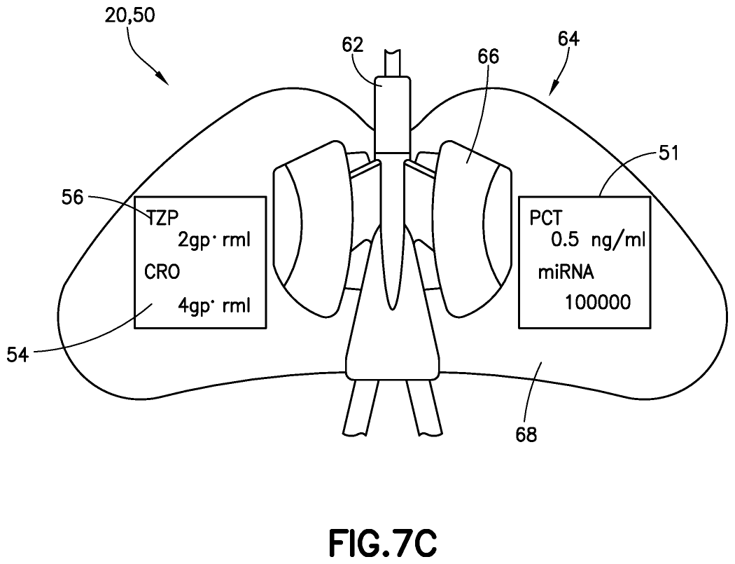

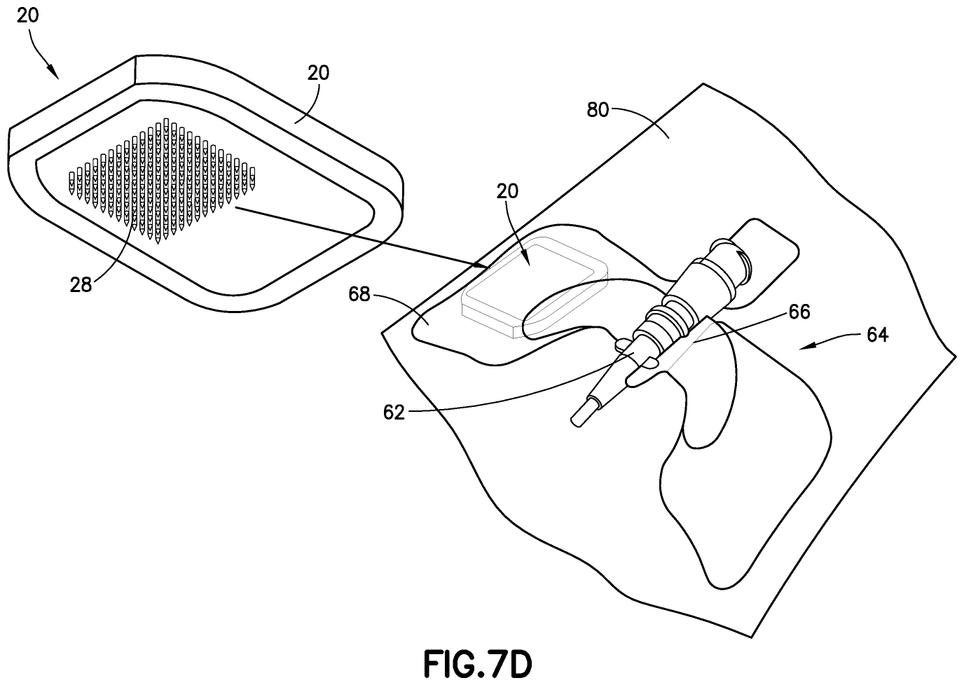

Referring to FIGS. 7A-7C an SMD 20 and SMS 50 in the form of a catheter care device 64 is shown according to non-limiting embodiments. The SMD 20 may be integrated with commercial catheter care devices 64, such as a StatLock device available from Becton, Dickinson and Company. A catheter 62 may be used with a patient to allow the passage of fluids and/or to distend body passages. The catheter care device 64 may secure the catheter 62 to the patient's skin once the catheter 62 is inserted into the patient to prevent the catheter from being easily removed or from being jostled. The catheter care device 64 may comprise a care mechanism 66 configured to engage with a component of the catheter 62 to secure the catheter 62 to the catheter care device 64. Further, the catheter care device 64 may comprise a band 68 connected to the care mechanism 66. The band 68 is adapted to be secured to an appendage of the patient in the area in which the catheter 62 has been inserted into the patient. In some non-limiting examples, the band 68 may be configured to wrap around an appendage (e.g., arm, leg, etc.) to secure the catheter care device 64 to the patient.

With continued reference to FIGS. 7A-7C, the catheter care device 64 may comprise the SMD 20 and comprise the computing device 51 so as to form the SMS 50. The SMD 20 may comprise the patch 22 having the microneedles 28 configured to penetrate the skin of the patient when the catheter care device 64 is secured to the patient. The SMD 20 may be integrated into the band 68 (e.g., an underside thereof) of the catheter care device 64 so that the microneedles 28 penetrate the patient's skin when the catheter care device 64 is secured to the patient. The SMD 20 may comprise the microfluidic device 60 to flow the ISF 32 to the relevant portion of the sensor 30. The catheter care device 64 may comprise the battery 58 to power the sensor 30 and/or the computing device 51.

The computing device 51 may be integrated into the band 68 of the catheter care device 64, although the computing device 51 may be integrated into other locations of the catheter care device 64 or may be separate from the catheter care device 64. The computing device 51 may comprise the display 54 to display biomarker data communicated to the computing device 51 from the SMD 20.

Referring to FIG. 7D, an SMD 20 in the form of a catheter care device 64 is shown according to non-limiting embodiments. The SMD 20 may be identical to the SMD 20 described in connection with FIGS. 7A-7C except as follows. The SMD 20 from FIG. 7D may not comprise an integrated computing device 51. The SMD 20 may communicate the biomarker data to a computing device 51 separate from the SMD 20, such as over a short-range wireless communication connection.

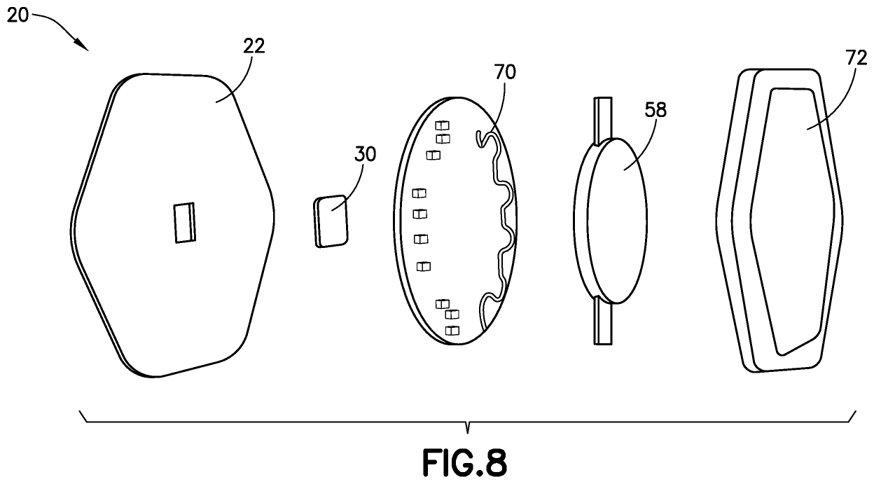

Referring to FIG. 8 an SMD 20 in the form of a skin patch is shown according to non-limiting embodiments. The SMD 20 may comprise the patch 22 and the sensor 30 as previously described. The patch 22 may be configured to be adhered to the skin of a patient to be worn as a skin patch. The patch 22 may comprise the microneedles 28 (not shown) as previously described. The SMD 20 may comprise electronic components 70 in electrical communication with the sensor 30 (which has a microneedle array on the other side). The electronic components 70 may process the data from the sensor 30. The electronic components 70 may communicate with the computing device 51 (not shown), such as by sending the signal comprising the biomarker data 56. The SMD 20 may comprise the battery 58 to power the sensor 30 and/or the electronic components 70. The SMD 20 may comprise an enclosure 72 configured to cover the components of the SMD 20 and to protect them from damage. For example, the enclosure 72 may protect the battery 58, the electronic components 70, the sensor 30, and the patch 22. The skin patch may be any other bandage-type device or skin dressing adapted to be affixed to the patient's skin.

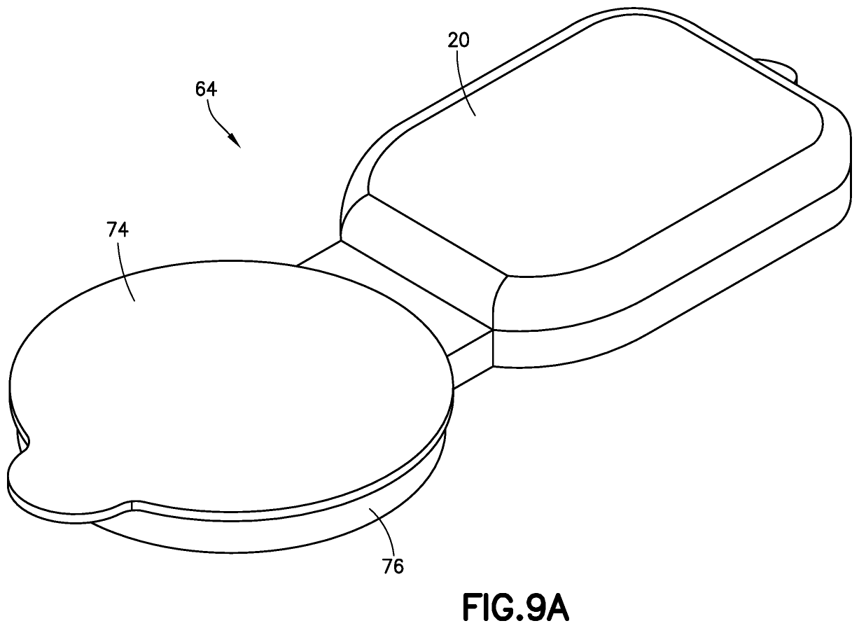

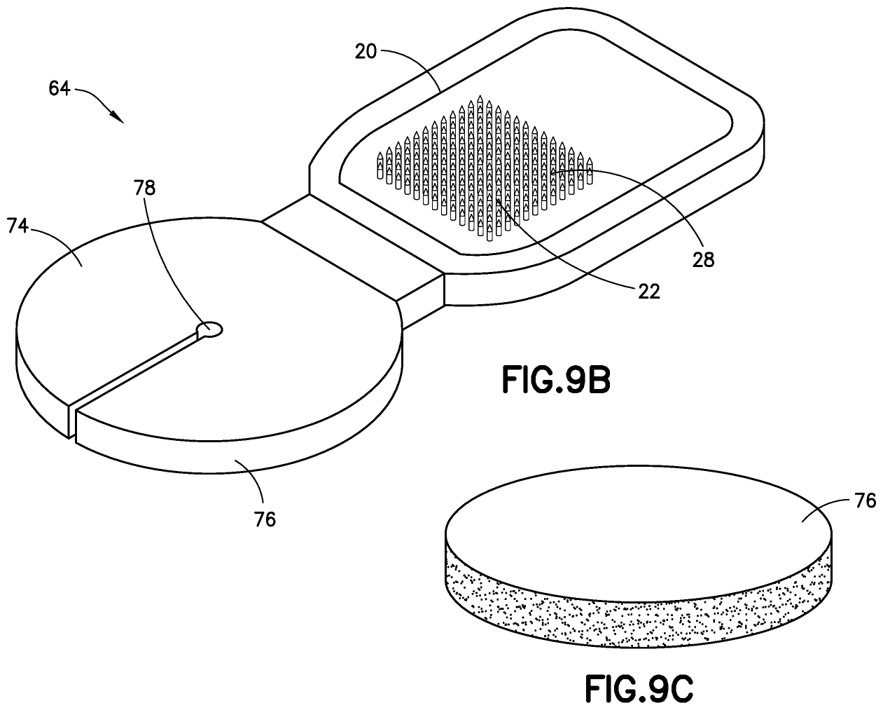

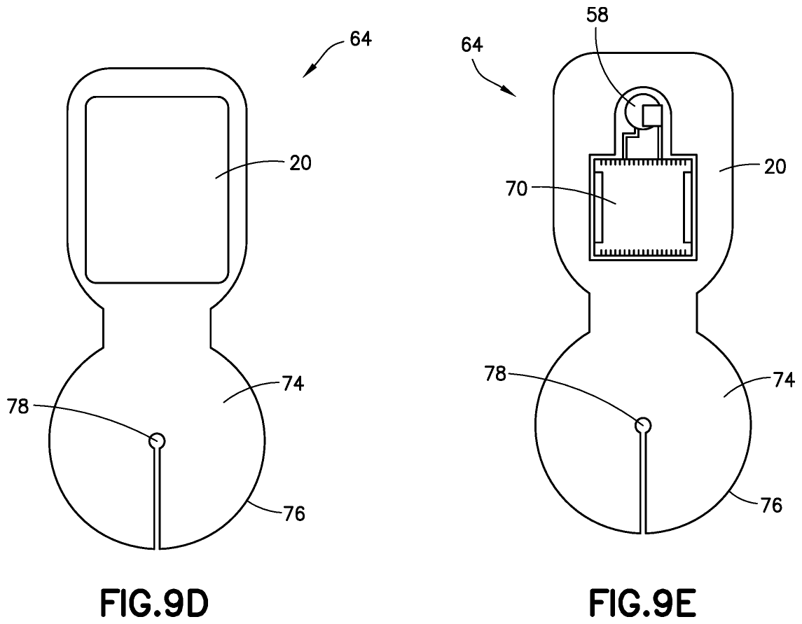

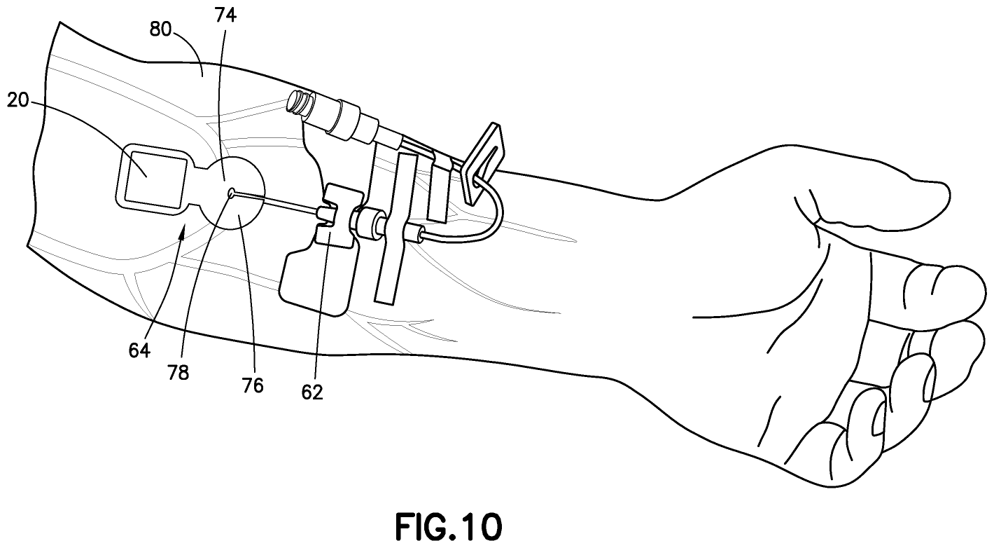

Referring to FIGS. 9A-9E, a SMD 20 in the form of a catheter care device 64 and/or catheter insertion site protection device and/or a skin patch is shown. The catheter care device 64 may comprise the SMD 20 and a catheter care portion 74 (e.g., anti-microbial and/or hemostasis performance of the catheter insertion site). The SMD 20 may comprise the patch 22 and the microneedles 28 as previously described. The SMD 20 may comprise the sensor 30 (not shown), the battery 58, and the electronic components 70 as previously described. The microneedles 28 may penetrate the user's skin when the catheter care device 64 is adhered to the patient.

With continued reference to FIGS. 9A-9E, the catheter care device 64 may comprise the catheter care portion 74, which is the portion of the catheter care device 64 which co-acts with the catheter 62 (not shown) to arrange the catheter 62 with respect to the patient. The SMD 20 may be integrated with commercial catheter care devices 64, such as the GuardIVa device available from Becton, Dickinson and Company. The care portion 74 may comprise a care pad 76. The care pad 76 may be made of any suitable material for taking care of the catheter insertion site for the patient. For example, the care pad 76 may comprise a foam to control/absorb exudate oozing out of insertion site loaded with an antimicrobial agent to protect the insertion site against microorganisms and a hemostasis agent to slow down the bleeding from the catheter insertion site. In some non-limiting examples, the hemostatic agent of the care pad 76 is formed from cellulose, such as a micro-dispersed, oxidized cellulose. The care pad 76 may comprise an absorbent material and may be incorporated with chlorhexidine gluconate (CHG) or other disinfectant and/or antiseptic. The disinfectant and/or antiseptic may disinfect that area of the patient in which the catheter 62 is inserted. As shown in FIGS. 9B, 9D, and 9E, the care pad 76 may define a slit 78. The slit 78 may receive the catheter 62 to enable the care pad 76 to take care of the catheter 62.

Referring to FIG. 10, a catheterized patient having the catheter taken care of by the catheter care device 64 from FIGS. 9A-9E is shown. In the non-limiting example from FIG. 10, the catheter 62 is inserted into the arm of a patient 80. The catheter 62 may be threaded through the slit 78 of the care pad 76 in the care portion 74 of the illustrated catheter care device 64. By this arrangement, the care portion 74 may take care of the catheter 62 to the arm of the patient 80. The catheter care device 64 also comprises the SMD 20 (having the patch 22, microneedles 28, and sensor 30—not shown). Thus, this catheter care device 64 may serve the dual function of caring for the catheter 62 to the patient 80 and monitoring the patient 80 for sepsis or the onset of sepsis. The microneedles 28 may collect ISF 32 from the patient 80 to monitor sepsis or the onset thereof. The microneedles 28 do not collect blood from the patient 80.

Once applied to the patient's 80 skin, the SMD 20 may continuously monitor biomarker levels of the patient for the duration that the SMD 20 is adhered to the patient's 80 skin. The SMD 20 may obtain fast results for determining biomarker levels, such as in less than 60 minutes, less than 30 minutes, less than 15 minutes, or less than 1 minute, such as less than 60 or 30 seconds.

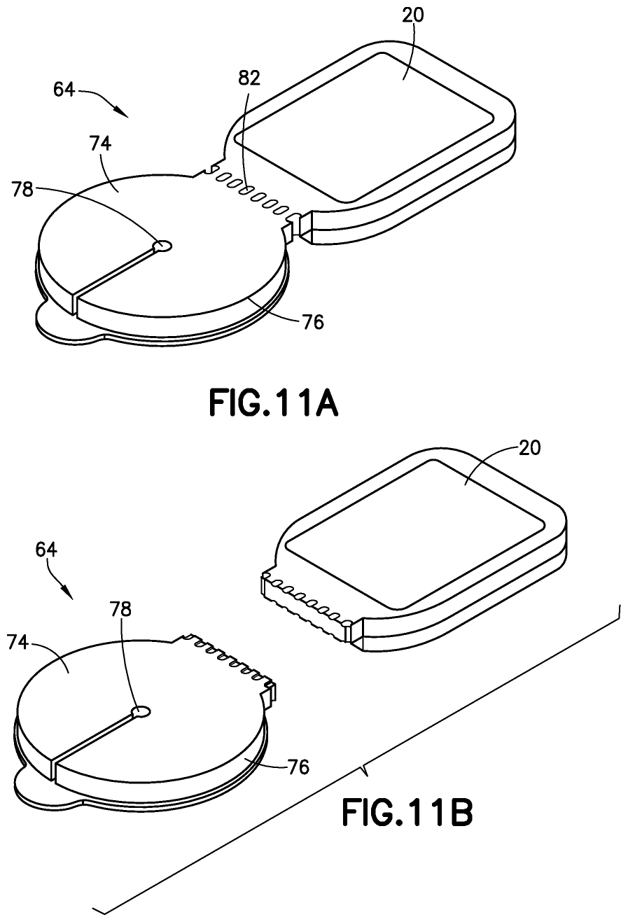

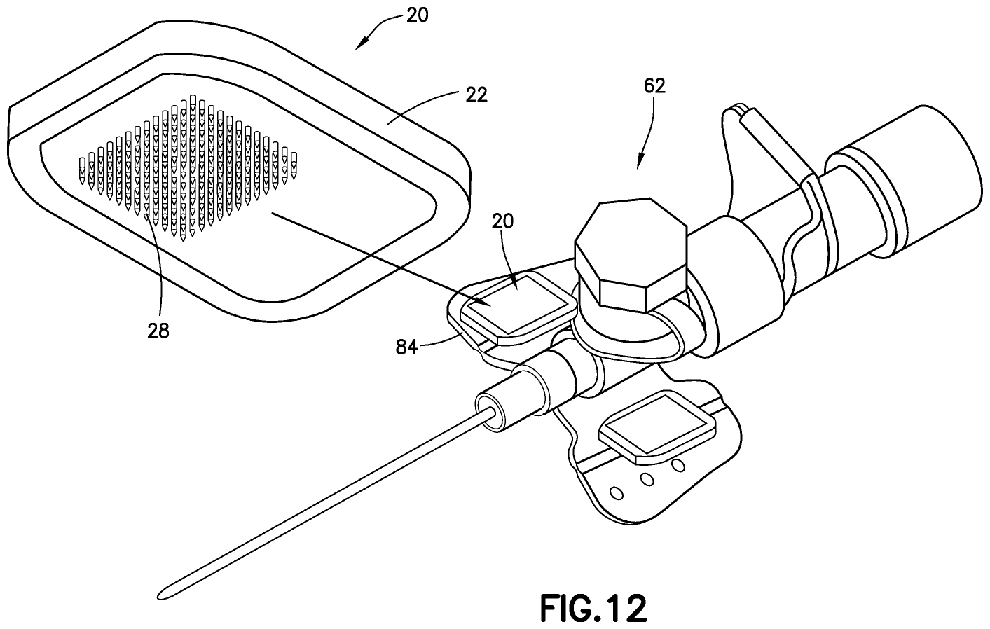

Referring to FIGS. 11A-11B, a SMD 20 in the form of a catheter care device 64 and/or catheter insertion site protection device and/or a skin patch is shown according to non-limiting embodiments. The catheter care device 64 from FIGS. 11A-11B is identical to the catheter care device shown in FIGS. 9A-9E except as follows. Like in FIGS. 9A-9E, the catheter care device 64 may have a SMD 20 and a care portion 74 as a single component such that the catheter care device 64, when adhered to a patient, serves the dual function of caring for the catheter 62 and monitoring the patient for sepsis or the onset of sepsis. However, the catheter care device 64 may further comprises a perforation 82. The perforation 82 may be positioned between the SMD 20 and the care portion 74. In FIG. 11A, the perforation 82 is not broken, such that the catheter care device 64 may be used for its dual functions. However, in FIG. 11B, the perforation 82 has been broken such that the SMD 20 and the care portion 74 are separate components. Once the perforation 82 has been broken, the care portion 74 may be adhered to the patient as a catheter care device 64, while the SMD 20 may be adhered to a different area of the patient to monitor the patient for sepsis or the onset of sepsis. Including the perforation 82 may enable a user to arrange the securing/care portion 74 and the SMD 20 on different areas of the patient.

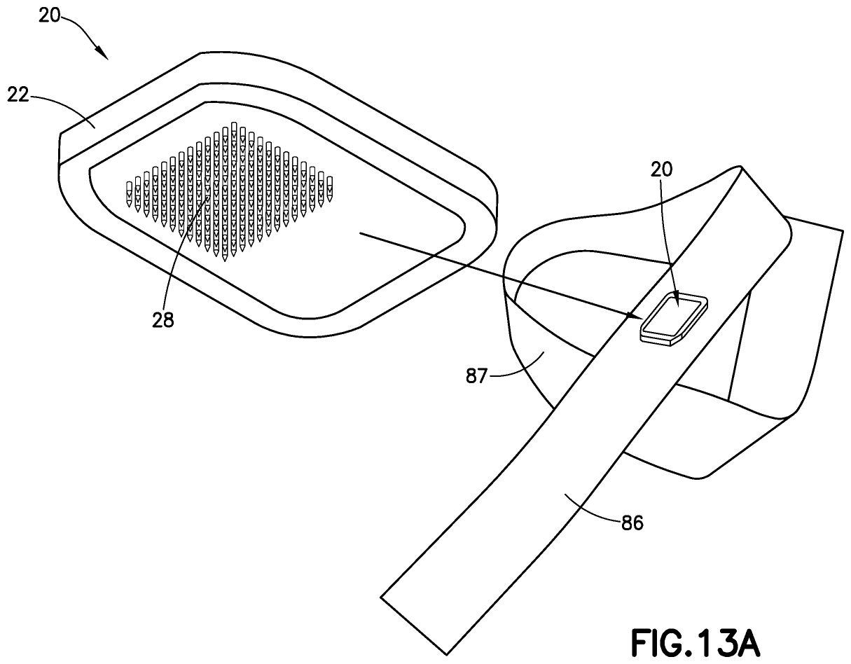

Referring to FIG. 12, a catheter 62 having an SMD 20 is shown according to non-limiting embodiments. While similar to the component shown in FIG. 7D, the catheter 62 shown in FIG. 12 is different as it does not include a catheter securing/care device 64. Instead, the SMD 20 is integrated directly into a catheter component 84 of the catheter 62. In this way, the catheter 62 itself includes a means to monitor sepsis or the onset thereof without the need for a catheter care device 64. The microneedles 28 from the SMD 20 may penetrate the patient's skin when the catheter 62 is inserted into the patient.

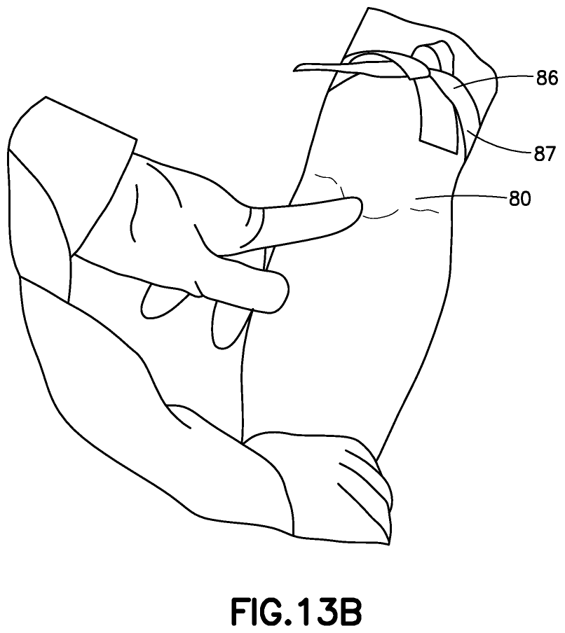

Referring to FIGS. 13A-13B, a SMD 20 in the form of a tourniquet 86 is shown according to non-limiting embodiments. The tourniquet 86 may be applied to an appendage (e.g., arm, leg, etc.) of the patient 80 to apply pressure thereto in order to stop the flow of blood. The SMD 20 may be attached to a band 87 of the tourniquet 86. When the tourniquet 86 is applied to the patient 80, it may be applied in such a way that the microneedles 28 of the SMD 20 penetrate the skin of the patient 80, so as to monitor sepsis or the onset thereof. When the tourniquet 86 is removed, the SMD 20 may be left on the patient's 80 skin (by detaching from the tourniquet 86) to enable continuous monitoring.

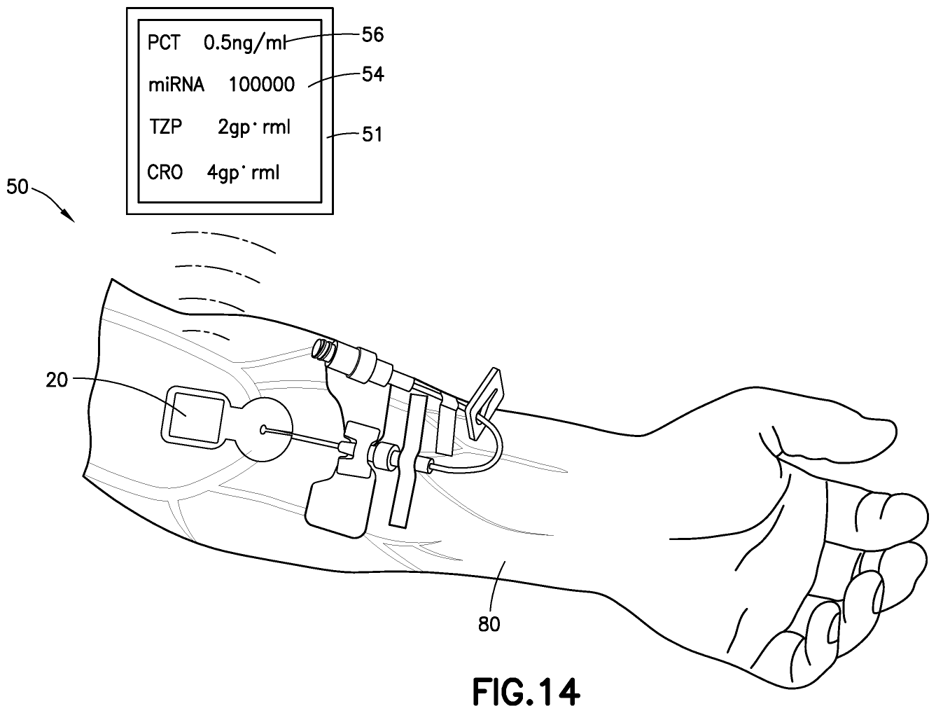

Referring to FIG. 14, an SMS 50 is shown according to non-limiting embodiments. The SMS 50 may include the SMD 20 in electrical communication with the computing device 51. As shown in FIG. 14, the SMD 20 may communicate a message to the computing device 51 to cause the computing device to display biomarker data 56 (e.g., a level of the biomarker 34) on the display 54. The SMD 20 may be connected to the computing device 51 by a wired (not shown) connection to communicate therewith, or the SMD 20 may wirelessly communicate with the computing device 51. The SMD 20 may be configured to communicate with the computing device 51 over a short-range wireless communication connection (e.g., an NFC communication connection, an RFID communication connection, a Bluetooth® communication connection, etc.).

The present disclosure is also directed to a method for monitoring sepsis. The method may include adhering the SMD to the skin of the patient by adhering the second side to the skin such that the plurality of microneedles penetrate the skin to a depth sufficient to collect ISF. The method may include collecting, via the plurality of microneedles, the ISF from the patient and flowing the ISF to the sensor. The sensor may detect a biomarker from the ISF, the biomarker indicating a sepsis condition or an onset of the sepsis condition.

From contact with the ISF, the sensor may determine the level of the relevant biomarker. The sensor may transmit the signal to the computing device indicating the detected level of the relevant biomarker. In response to receiving the signal, the computing device may display the level of the biomarker. In response to the level of the biomarker satisfying a threshold, treatment may be initiated targeted at treating the patient for sepsis.

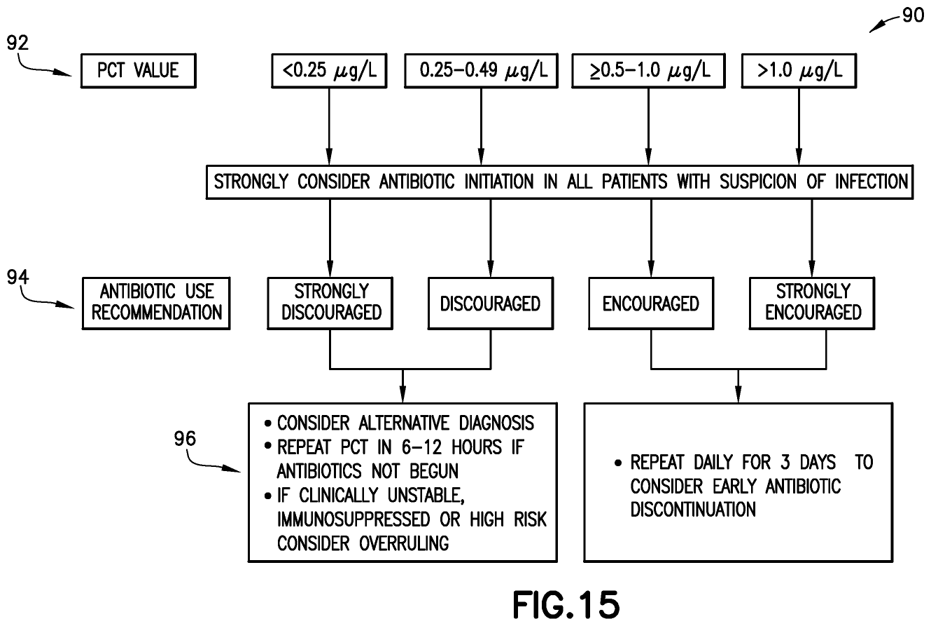

Referring to FIG. 15, a sepsis treatment plan 90 is shown according to non-limiting embodiments. The sepsis treatment plan 90 may comprise thresholds 92 associated with biomarkers relevant to detecting sepsis or the onset thereof. The thresholds 92 may correspond to an amount and/or concentration of a particular biomarker. For example, the thresholds 92 may specify a range of an amount or concentration for a relevant protein (e.g., PCT or IL-6) and/or a relevant nucleic acid (e.g., miRNA-223). In response to the level of a relevant biomarker satisfying a threshold, a treatment may be initiated to treat the patient for sepsis. The treatment may include an automated action (e.g., automated delivery of at least one medicament), or an action initiated by a healthcare professional. The amount and/or the timing of a medicament delivered to the patient may be altered (started, stopped, adjusted) based on the biomarker data collected by the SMD. A proportional-integral-derivative (PID) controller and/or an iterative learning controller (ILC) may automatically alter the delivery of the medicament. In response to the level of a relevant biomarker satisfying a threshold, the healthcare professional may be automatically notified of the condition.

With continued reference to FIG. 15, the treatment plan 90 may comprise treatment recommendations 94. Treatment recommendations 94 may correspond to thresholds 92 such that if a certain threshold 92 is satisfied, a corresponding treatment recommendation 94 is provided. For example, the treatment recommendation 94 may comprise administering or refraining from administering an effective antibiotic treatment depending on the threshold 92 currently satisfied by the patient's relevant biomarker level. The treatment plan 90 may also comprise treatment details 96 corresponding to each treatment recommendation 94. The treatment details 96 may comprise specific instructions and/or recommendations for the healthcare professional based on the threshold 92 currently satisfied by the patient's relevant biomarker level.

Although the present disclosure has been described in detail for the purpose of illustration based on what is currently considered to be the most practical and preferred embodiments, it is to be understood that such detail is solely for that purpose and that the present disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

The invention claimed is:

1. A sepsis monitoring device, comprising:
a patch having a first side and an opposing second side, wherein the second side is adapted to adhere to human skin;
a plurality of microneedles arranged on the second side of the patch and configured to penetrate the human skin to a depth sufficient to collect interstitial fluid when the patch is adhered to the human skin; and
a sensor in fluid communication with the plurality of microneedles and configured to detect a biomarker indicating a sepsis condition or an onset of the sepsis condition when contacted with the interstitial fluid collected by the plurality of microneedles.

2. The device of claim 1, wherein the biomarker comprises at least one of a protein, a nucleic acid, and/or some combination thereof.

3. The device of claim 2, wherein the nucleic acid comprises miRNA.

4. The device of claim 2, wherein the protein comprises a C-reactive protein (CRP), Procalcitonin (PCT), Interleukin (IL)-6, and/or some combination thereof.

5. The device of claim 1, wherein the biomarker comprises a combination of miRNA-223 and Procalcitonin (PCT).

6. The device of claim 1, wherein each of the plurality of microneedles is hollow or solid and has a length of up to 2,000 microns.

7. The device of claim 1, wherein the sensor comprises an electrochemical electrode comprising a conductive electrode comprising sensing materials.

8. The device of claim 7, wherein the sensing materials comprise a hetero-bifunctional carboxyl acid- and sulfhydryl-reactive polyethylene glycol crosslinker capable of linking to the biomarker.

9. The device of claim 1, wherein the sensor is configured to detect a level of the biomarker and transmit a signal to a computing device, the signal indicating the level of the biomarker.

10. The device of claim 9, wherein the signal is configured to cause the computing device to display the level of the biomarker.

11. The device of claim 9, wherein the sensor is configured to transmit the signal to the computing device over a short-range wireless communication connection.

12. The device of claim 1, wherein the device comprises a wearable electronic device.

13. The device of claim 1, wherein the device comprises at least one of a skin patch, a catheter care device, a catheter, and/or a tourniquet.

14. The device of claim 1, wherein the plurality of microneedles is formed from a hydrogel polymer, metal, plastic, silicon, elastomer, or combination thereof.

15. The device of claim 1, wherein the device is configured to monitor for the sepsis condition or the onset of the sepsis condition using the interstitial fluid from a dermal layer of skin and without a blood sample.
